(19) United States
(12) Patent Application Publication
(10) Pub. No.: US 2026/0224232 A1
(43) Pub. Date: Aug. 6, 2026

(54) MULTI-CAVITY ASPIRATION DEVICE

(71) Applicant: MicroVention, Inc., Aliso Viejo, CA (US)

(72) Inventor: Belete Degaga, Aliso Viejo, CA (US)

(73) Assignee: MicroVention, Inc., Aliso Viejo, CA (US)

(21) Appl. No.: 19/152,909

(22) PCT Filed: Feb. 2, 2024
(86) PCT No.: PCT/US2024/014229

§ 371 Date: Jul. 31, 2025

Related U.S. Application Data
(60) Provisional application No. 63/482,969, filed on Feb. 2, 2023.

(51) Int. Cl. A61B 17/22; A61B 17/00; A61B 90/00; A61M 25/00

(52) U.S. Cl. CPC A61B 17/22; A61B 90/39; A61M 25/0068; A61B 2017/00544; A61B 2017/00778; A61B 2017/22079; A61B 2090/3966; A61B 2217/005; A61M 2202/0014; A61M 2202/06; A61M 2205/32

(57) ABSTRACT

A multi-cavity aspiration device for more efficiently aspirating multiple obstructions, clots, thrombi, emboli, or the like in a single aspiration stroke. The multi-cavity aspiration device may include a tubular member having an inner circumference. One or more cavities may be formed or cut into the inner circumference of the tubular member so as to increase surface area without affecting structural integrity. The one or more cavities may be positioned radially about the inner circumference of the lumen. The one or more cavities may be tapered proximally and terminate at a point. The one or more cavities may be exposed to the internal lumen of the tubular member.

### RELATED APPLICATIONS

This application claims benefit of and priority to U.S. Provisional Application Ser. No. 63/482,969 filed Feb. 2, 2023 entitled Multi-Cavity Aspiration Device, which is hereby incorporated herein by reference in its entirety.

### BACKGROUND

Aspiration devices are commonly used in a wide range of medical procedures which would benefit from application of a suction force in a body cavity or vessel. For example, aspiration catheters are commonly used for the removal of thrombi, emboli, debris, and/or other particles from various anatomies, such as in the coronary or peripheral arteries, via percutaneous suction.

An aspiration catheter of varying internal diameter may be advanced to a thrombosed segment, with the distal end of the aspiration catheter being positioned proximal to the clot. A negative pressure may then be applied to the aspiration catheter so as to suction the clot into the catheter for removal from the body. However, in some circumstances, multiple aspiration strokes may be necessary to successfully remove a clot.

It would be beneficial to provide an aspiration device capable of extracting multiple thrombi having variable geometries and/or dimensions in a single aspiration stroke.

### SUMMARY

A multi-cavity aspiration device is described for extracting one or more thrombi having variable geometries and/or dimensions (e.g., sizes) in a single aspiration stroke.

In some aspects, the multi-cavity aspiration device may comprise a tubular member having at least one internal lumen. The internal lumen may include one or more cavities, such as internal grooves, channels, opening, slots, or the like, to allow for the quick entry of a large volume of emboli in a single aspiration stroke.

In some aspects, the one or more cavities may be tapered in a proximal direction such that the one or more cavities are wider at a distal end and narrower at a proximal end.

In some aspects, the one or more cavities may not be tapered and thus may have a uniform width along their lengths.

In some aspects, the one or more cavities may converge to terminate at a point.

In some aspects, the one or more cavities may comprise a substantially conical shape.

In some aspects, the one or more cavities may comprise one or more scallops formed in an inner circumference of a lumen.

In some aspects, the one or more cavities may extend for only a fraction (e.g., 10% or less) of a length of the tubular member.

In some aspects, a plurality of cavities may be formed in an inner wall of the lumen of a tubular member, such as an aspiration catheter.

In some aspects, the plurality of cavities may be radially positioned about an inner circumference of the tubular member.

In some aspects, each of the plurality of cavities may be substantially the same length and width.

In some aspects, one or more of the cavities may have different dimensions (length, width, or the like) than one or more of the remaining cavities.

In some aspects, each of the cavities may be an equidistance with respect to the remaining cavities.

In some aspects, four cavities may be utilized, with each of the cavities being positioned at 90 degree intervals around an inner circumference of the tubular member.

In some aspects, the tubular member may include a radiopaque marker band positioned proximally with respect to a terminal end of the cavities.

In some aspects, the tubular member may comprise an aspiration catheter. The aspiration catheter may include one or more internal lumens.

In some aspects, the one or more internal lumens may comprise a guidewire lumen and an aspiration lumen. The aspiration lumen may be larger (e.g., wider) than the guidewire lumen.

In some aspects, a negative pressure causing a suction force may be applied to the aspiration lumen for removing the clot, debris, thrombus, embolus, or other particle.

In some aspects, the negative pressure may be applied manually, such as by a syringe, or automatically, such as by a pump.

In some aspects, the multi-cavity aspiration device may be applied to neuro and peripheral applications.

### BRIEF DESCRIPTION OF THE DRAWINGS

The following figures are included to illustrate certain aspects of the present disclosure and should not be viewed as exclusive examples. The subject matter disclosed is capable of considerable modifications, alterations, combinations, and equivalents in form and function, as will occur to one having ordinary skill in the art and having the benefit of this disclosure. Reference is being made to the accompanying drawings, in which:

FIG. 1A is a frontal perspective view of a multi-cavity aspiration device, according to an example of the present disclosure.

FIG. 1B is a frontal perspective view of a multi-cavity aspiration device, according to an example of the present disclosure.

FIG. 2 is a partial perspective view of a multi-cavity aspiration device, according to an example of the present disclosure.

FIG. 3 is a partial side view of a multi-cavity aspiration device, according to an example of the present disclosure.

FIG. 4 is an end view of a multi-cavity aspiration device, according to an example of the present disclosure.

FIG. 5 is a sectional view taken along line 5-5 of FIG. 3, according to an example of the present disclosure.

FIG. 6 is a sectional view taken along line 6-6 of FIG. 3, according to an example of the present disclosure.

FIG. 7 is an end view of another example multi-cavity aspiration device, according to an example of the present disclosure.

FIG. 8 is a partial perspective view of the multi-cavity aspiration device of FIG. 7, according to an example of the present disclosure.

FIG. 9 is an end view of another example multi-cavity aspiration device, according to an example of the present disclosure.

FIG. 10 is a partial side view of the multi-cavity aspiration device of FIG. 9, according to an example of the present disclosure.

FIG. 11 is an end view of another example multi-cavity aspiration device, according to an example of the present disclosure.

FIG. 12 is a partial side view of the multi-cavity aspiration device of FIG. 11, according to an example of the present disclosure.

FIG. 13 is an end view of another example multi-cavity aspiration device, according to an example of the present disclosure.

FIG. 14 is a partial side view of the multi-cavity aspiration device of FIG. 13, according to an example of the present disclosure.

FIG. 15 is an end view of another example multi-cavity aspiration device, according to an example of the present disclosure.

FIG. 16 is a partial side view of the multi-cavity aspiration device of FIG. 15, according to an example of the present disclosure.

### DETAILED DESCRIPTION

Specific examples of the disclosure will now be described with reference to the accompanying drawings. This disclosure may, however, be embodied in many different forms and should not be construed as limited to the examples set forth herein; rather, these examples are provided so that this disclosure will be thorough and complete, and will fully convey the scope of the disclosure to those skilled in the art. The terminology used in the detailed description of the examples illustrated in the accompanying drawings is not intended to be limiting of the disclosure. In the drawings, like numbers refer to like elements.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

Disclosed herein are various examples of a multi-cavity aspiration device which may be utilized for more rapidly and efficiently extracting one or more thrombi, emboli, particles, clots, and the like having various sizes and/or geometries in a single aspiration stroke. The systems and methods shown and/or described herein may be utilized for a wide range of purposes in a wide range of anatomies, and thus should not be construed as limited in scope by the examples disclosed herein.

As a non-limiting example, the systems and methods shown and/or described herein may be utilized for the removal of clots or the like from anatomies such as the coronary or peripheral arteries via percutaneous suction. By way of example and without limitations, the systems and methods shown and/or described herein may be applied to neuro and peripheral applications.

The systems and methods shown and/or described herein may be particularly suited for use with turbulent flows. Turbulent flows may be present where the flow pathway becomes disorganized, layers break formation, and/or eddy currents are formed. In arteries, for example, turbulent blood flow can occur where atherosclerotic plaques narrow and vary the vessel lumen, where blood vessels branch, or where aneurysms are encountered. The pronounced bend in the ascending aorta may also create a turbulent flow. Turbulent flow may generate sound, create murmurs, carotid bruits, and other audible diagnostic clues.

Risk factors that increase turbulence, such as plaque build-up or hardening of the arterial walls, may serve to increase vascular resistance (i.e., systematic vascular resistance or SVR) and thereby result in increased blood pressure, reduced blood flow, and/or an increased workload for the heart. The multi-cavity aspiration device may improve the odds of capturing irregular-shaped emboli in such turbulent flows.

The use of one or more cavities on an internal wall of a lumen of a tubular member may allow the lumen to be effectively increased in diameter while maintaining wall structure and structural integrity of the tubular member. By using a lumen having an increased effective diameter, aspiration of a clot or other obstruction may be more efficiently performed with a stronger suction force. Inclusion of the cavities shown and/or described herein may allow for such an increased diameter of the lumen while minimizing or reducing the risk of lumen collapse due to a lack of structural integrity of the tubular member.

The multi-cavity aspiration device may comprise various types of medical devices used for aspiration of clots, such as but not limited to aspiration catheters. The multi-cavity aspiration device may comprise a tubular member having at least one internal lumen. An inner circumference of the internal lumen may include one or more cavities, such as internal grooves, channels, openings, slots, elongated openings, scallops, or the like, to allow for the quick entry of a large volume of emboli, or emboli having different sizes/geometries, in a single aspiration stroke and thereby significantly improve efficiency of clot retrieval.

The use of the one or more cavities on an internal wall of a lumen of a tubular member may also aid in breaking up or otherwise disrupting (e.g., compressing) the shape of a clot or other obstruction such that the clot or other obstruction may be more efficiently or effectively suctioned into the tubular member. By way of example, the shape of the one or more cavities may form leading edges or the like which aid in breaking up or otherwise disrupting the shape of the clot or other obstruction at the distal mouth of the tubular member such that the clot or other obstruction may more easily fit within the lumen of the tubular member for suction and removal. Further, by disrupting the shape (e.g., the outer profile) of the clot or other obstruction, fewer aspiration strokes may be needed to effectively suction the clot or other obstruction.

The tubular member may include a single internal lumen, or multiple internal lumens. For example, the tubular member may include a first internal lumen for receiving a guidewire and a second internal lumen for aspiration. The second internal lumen may be larger in diameter than the first internal lumen.

The shape, size, orientation, and positioning of the one or more cavities may vary depending on various factors, such as but not limited to the target location in the patient's body and the type, volume, and/or size of the clot(s) being retrieved via aspiration. The one or more cavities may be tapered in a proximal direction such that the one or more cavities are wider at a distal end and narrower at a proximal end. The one or more cavities may have a uniform diameter and thus not be tapered along their lengths.

The one or more cavities may terminate to a point, such as at a proximal end of the one or more cavities. The one or more cavities may be open to the internal lumen, such as in a scalloped configuration. The one or more cavities may comprise a wide variety of shapes. Thus, the shapes of the one or more cavities illustrated in the figures should not be construed as limiting in scope. By way of example and without limitation, the one or more cavities may comprise elongated rectangular shapes, cylindrical shapes, semi-cylindrical shapes, elongated shapes, semi-circular shapes, circular shapes, oval shapes, semi-oval shapes, polygonal shapes, triangular shapes, cone shapes, pentagonal shapes, hexagonal shapes, septagonal shapes, octagonal shapes, and the like.

In some examples, the one or more cavities may extend proximally from the distal mouth (e.g., the distal tip) of the tubular member. The length of each cavity may vary in different examples. Further, in some examples including a plurality of cavities, each of the cavities may comprise the same length. In other examples including a plurality of cavities, one or more of the cavities may have a different length than one or more of the remaining cavities.

The length of the one or more cavities as a ratio of the overall length of the tubular member may vary in different examples. The one or more cavities may extend for only a portion of a length of the tubular member, such as less than 10%, less than 20%, less than 30%, less than 40%, less than 50%, less than 60%, less than 70%, less than 80%, less than 90%, or less than 100% of the length of the tubular member. In some examples, however, the one or more cavities may extend for the full length of the tubular member (e.g., from its distal end to its proximal end).

The one or more cavities may be formed in an inner wall or inner circumference of the internal lumen of the tubular member. Where the tubular member includes multiple internal lumens, the one or more cavities may be positioned within the internal lumen meant for aspiration (i.e., the internal lumen to which a negative suction force is applied).

Where multiple cavities are utilized, each of the cavities may comprise substantially the same dimensions (e.g., length and/or width). However, one or more of the cavities may have different dimensions than one or more of the remaining cavities. The cavities may be evenly spaced about the internal circumference of the lumen, such as in 90-degree intervals where four cavities are utilized, or may be arranged in other configurations wherein some of the cavities are closer to each other than other cavities. Thus, in some examples, only a portion of the internal circumference of the lumen may include cavities, with the remaining portion of the internal circumference of the lumen not including any cavities. As an example, a first half (e.g., 180 degrees) of the internal circumference of the lumen may include one or more cavities and the remaining second half (e.g., the remaining 180 degrees) of the internal circumference of the lumen may include no such cavities.

The tubular member may include radiopaque features to aid in visualization with various imaging devices during use. For example, a radiopaque marker band may be positioned about an outer circumference of the tubular member, such as at or near a proximal termination point of the one or more cavities.

The inclusion of one or more cavities within an inner circumference of an internal lumen of a tubular member may affect the structural integrity of the tubular member, particularly for the portion of its length along which the one or more cavities extend. However, various methods may be utilized to counteract or eliminate such a reduction in structural integrity and thereby reinforce the tubular member so as to prevent collapse. The tubular member may itself be reinforced, such as by the use of internal coils or other structural members (e.g., ribs or the like) within the jacket or wall of the tubular member. Additionally or alternatively, a reinforcing band, such as a radiopaque marker band, may be positioned or secured over the tubular member, such as along the portion of the tubular member's length that the one or more cavities extend.

Aspiration pressure, such as a negative pressure or suction force, may be applied to the tubular member to aspirate a clot or other obstruction. The aspiration pressure may be applied manually, such as by a syringe, or automatically, such as by a pump.

In some examples, a medical device may comprise a catheter having a proximal portion, a distal portion, and a lumen therebetween. The catheter may have an inner diameter, an outer diameter, and a catheter sidewall therebetween. The catheter sidewall may include one or more longitudinal cavities at the distal portion of the catheter that are in fluid communication with the lumen and extend to a distal tip of the catheter.

Specific examples are described further below. However, it should be understood that any of the features from any of the examples can be mixed and matched with each other in any combination. Hence, the present disclosure should not be restricted to only these examples, but any broader combination thereof.

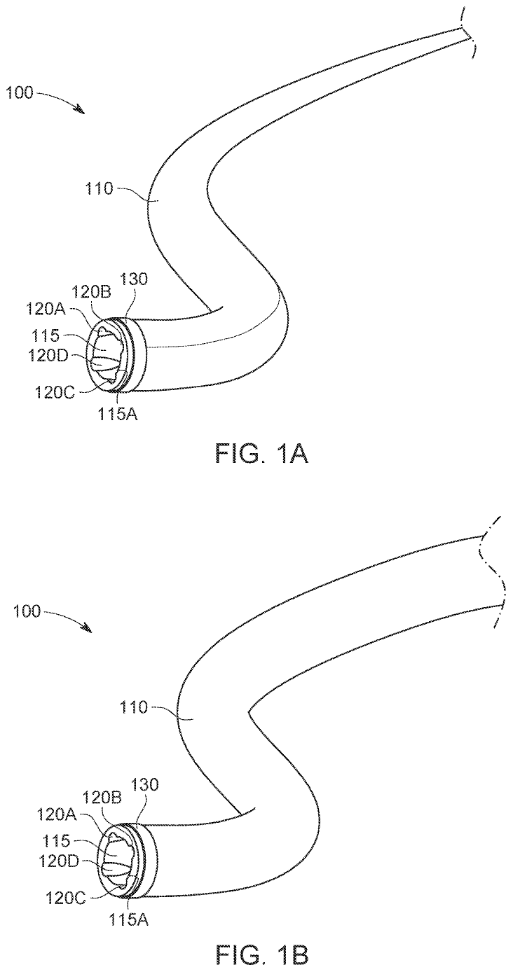

FIG. 1A is a frontal perspective view of a multi-cavity aspiration device 100, according to an example of the present disclosure. As shown, the multi-cavity aspiration device 100 may comprise a tubular member 110 having at least an aspiration lumen 115. One or more cavities 120A, 120B, 120C, 120D may extend proximally from a distal end of the inner circumference 115A of the aspiration lumen 115. In the example shown in FIG. 1A, the tubular member 110 as well as its aspiration lumen 115 may taper inwards in the proximal direction (e.g., the diameter or circumference of the tubular member 110 and aspiration lumen 115 may be gradually reduced in the proximal direction).

FIG. 1B is a frontal perspective view of a multi-cavity aspiration device 100, according to an example of the present disclosure. In the example shown in FIG. 1B, the tubular member 110 and aspiration lumen 115 may comprise a uniform diameter or circumference for an entire length of the tubular member 110 in some examples.

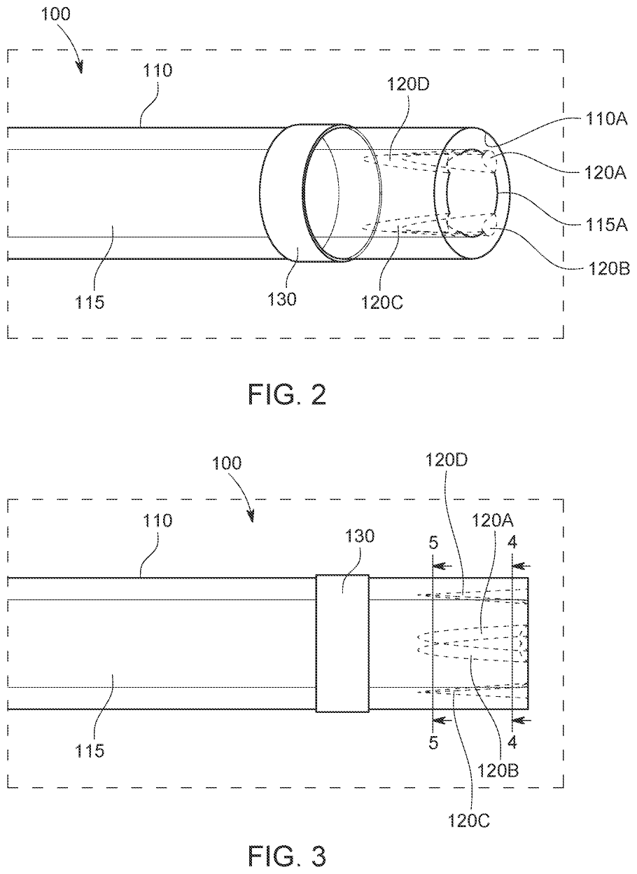

FIG. 2 is a partial perspective view of a multi-cavity aspiration device 100, according to an example of the present disclosure. FIG. 3 is a partial side view of a multi-cavity aspiration device 100, according to an example of the present disclosure. As shown, the multi-cavity aspiration device 100 may comprise a tubular member 110, such as an aspiration catheter, having a distal end 110A and at least one internal lumen 115 having an inner circumference 115A. In some examples, one or more cavities 120A, 120B, 120C, 120D may be positioned within the inner circumference 115A of the internal lumen 115 of the tubular member 110. In other words, the one or more cavities may be formed in the inner circumference 115A of the tubular member 110 which can increase the effective circumference of the lumen 115 of the tubular member 110. In some examples, a radiopaque marker band 130 may be positioned about an outer circumference of the tubular member 110, such as at or near a proximal termination point of the one or more cavities 120A, 120B, 120C, 120D.

In some examples, the one or more cavities 120A, 120B, 120C, 120D may each comprise the same shape, different shapes, or combinations of the same shape and different shapes. Thus, in some examples having a plurality of cavities 120A, 120B, 120C, 120D, each of the cavities 120A, 120B, 120C, 120D may comprise the same shape as each other. In other examples having a plurality of cavities 120A, 120B, 120C, 120D, one or more of the cavities 120A, 120B, 120C, 120D may comprise a different shape from one or more of the remaining cavities 120A, 120B, 120C, 120D.

In some examples, having a plurality of cavities 120A, 120B, 120C, 120D, the cavities 120A, 120B, 120C, 120D may comprise substantially the same dimensions (e.g., length and/or width), have different dimensions, or any combination thereof. Thus, in some examples having a plurality of cavities 120A, 120B, 120C, 120D, each of the cavities 120A, 120B, 120C, 120D may comprise the same dimensions as each other. In other examples having a plurality of cavities 120A, 120B, 120C, 120D, one or more of the cavities 120A, 120B, 120C, 120D may comprise different dimensions than one or more of the remaining cavities 120A, 120B, 120C, 120D.

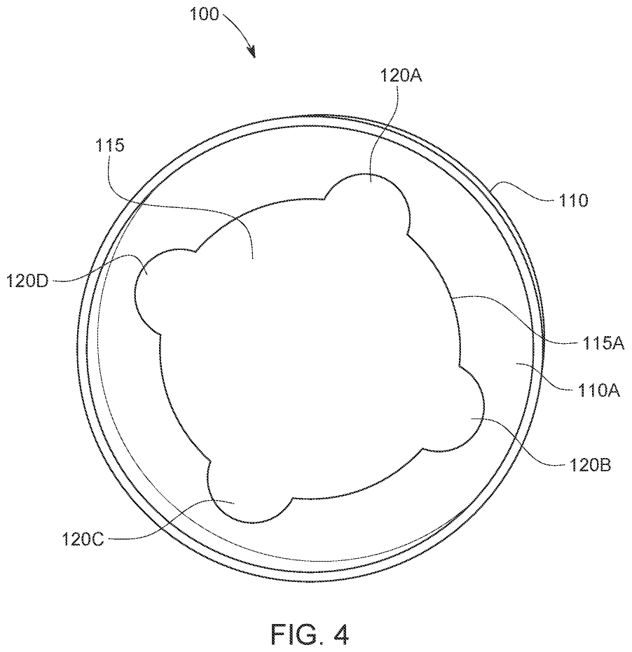

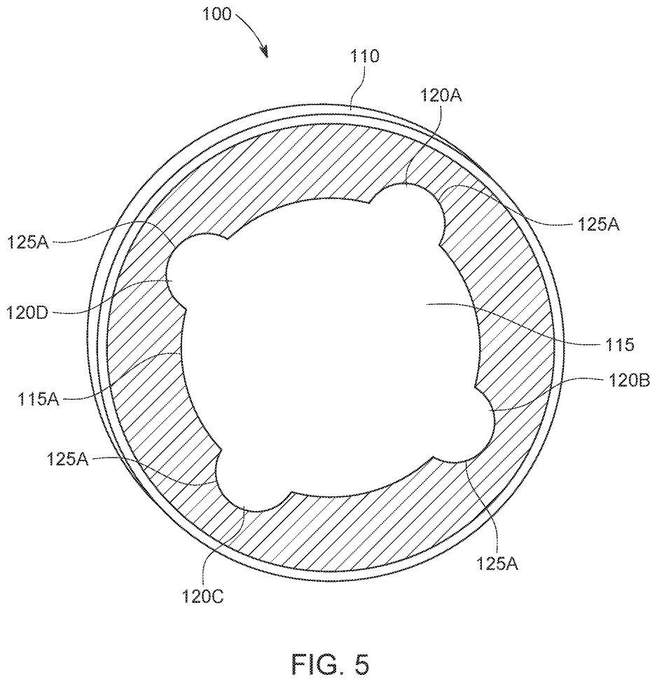

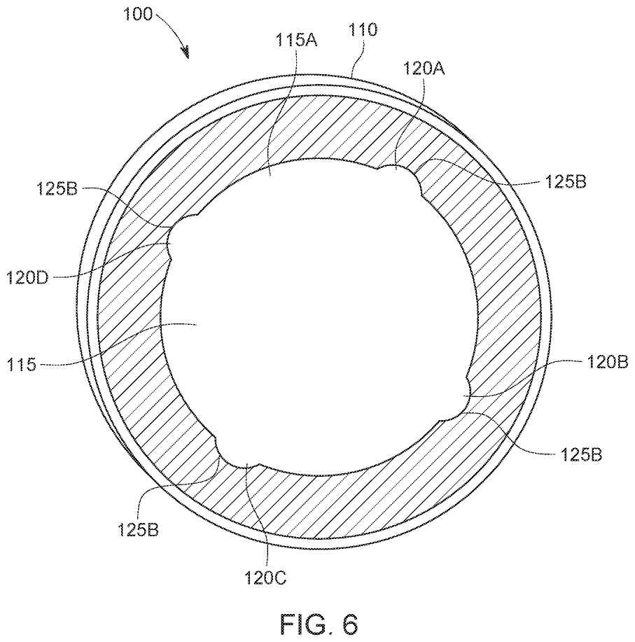

FIG. 4 is an end view of a multi-cavity aspiration device, according to an example of the present disclosure. FIG. 5 is a sectional view taken along line 5 -5 of FIG. 3, according to an example of the present disclosure. FIG. 6 is a sectional view taken along line 6 -6 of FIG. 3, according to an example of the present disclosure.

FIGS. 4-6 illustrate example cavities 120A, 120B, 120C, 120D formed within an inner circumference 115A of a lumen 115 of a tubular member 110 at or near its distal end 110A. In the illustrated examples, cavities 120A, 120B, 120C, 120D may comprise a first cavity 120A, a second cavity 120B, a third cavity 120C, and a fourth cavity 120D. Although the examples shown in the figures illustrate use of four cavities 120A, 120B, 120C, 120D, it should be appreciated that more (e.g., five or more) cavities 120A, 120B, 120C, 120D or less (e.g., three or less) cavities 120A, 120B, 120C, 120D may be utilized in different examples.

Each of the cavities 120A, 120B, 120C, 120D may be cut into or otherwise formed in the inner circumference 115A of the lumen 115 at or near the distal end 110A of the tubular member 110. The one or more cavities 120A, 120B, 120C, 120D may comprise various configurations, such as but not limited to internal grooves, channels, openings, slots, scallops, or the like. Generally, an inner face of each of the one or more cavities 120A, 120B, 120C, 120D may be exposed to (e.g., in fluid communication with) the internal lumen 115 of the tubular member 110 such as shown in the figures.

In some examples, the one or more cavities 120A, 120B, 120C, 120D may comprise a tapering width or diameter along their respective lengths, with the one or more cavities 120A, 120B, 120C, 120D either increasing or reducing in width or diameter in the proximal direction. In other examples, the one or more cavities may comprise a uniform width or diameter along their respective lengths.

In the example shown in FIGS. 5-6, the one or more cavities 120A, 120B, 120C, 120D may be proximally tapered such that a distal end 125A of each of the one or more cavities 120A, 120B, 120C, 120D is wider than a proximal end 125B of each of the one or more cavities 120A, 120B, 120C, 120D. The one or more cavities 120A, 120B, 120C, 120D may terminate to a point at their respective proximal ends 125B.

The positioning of the one or more cavities 120A, 120B, 120C, 120D, both with respect to the inner circumference 115A of the lumen 115 and with respect to each other, may vary in different examples. The one or more cavities 120A, 120B, 120C, 120D may be equally-spaced about the inner circumference 115A of the lumen 115. However, in some examples, some of the one or more cavities 120A, 120B, 120C, 120D may be closer to each other than others of the one or more cavities 120A, 120B, 120C, 120D. Where four cavities 120A, 120B, 120C, 120D are utilized, each of the cavities 120A, 120B, 120C, 120D may be arranged at 90-degree intervals such as shown in FIGS. 3-5. However, in some examples, the cavities 120A, 120B, 120C, 120D may be grouped together such as, e.g., by grouping multiple cavities 120A, 120B, 120C, 120D along a portion (e.g., 30 degrees, 45 degrees, 60 degrees, 75 degrees, 90 degrees, 105 degrees, 120 degrees, etc.) of the inner circumference 115A of the lumen 115, with the remaining portion of the inner circumference 115A of the lumen 115 not including any cavities 120A, 120B, 120C, 120D.

The number of cavities 120A, 120B, 120C, 120D utilized may also vary in different examples. In the example shown in FIGS. 4-6, four cavities 120A, 120B, 120C, 120D may be utilized. However, three or less, or five or more, cavities 120A, 120B, 120C, 120D may be utilized in some examples.

The dimensions of the cavities 120A, 120B, 120C, 120D may vary in different examples. The width of the wider distal ends of each of the cavities 120A, 120B, 120C, 120D may be greater or lesser than is shown in the examples illustrated in the figures. Further, the length of the cavities 120A, 120B, 120C, 120D as compared to a length of the tubular member 110 may vary in different examples, and thus should not be construed as limited in scope by the examples illustrated in the figures.

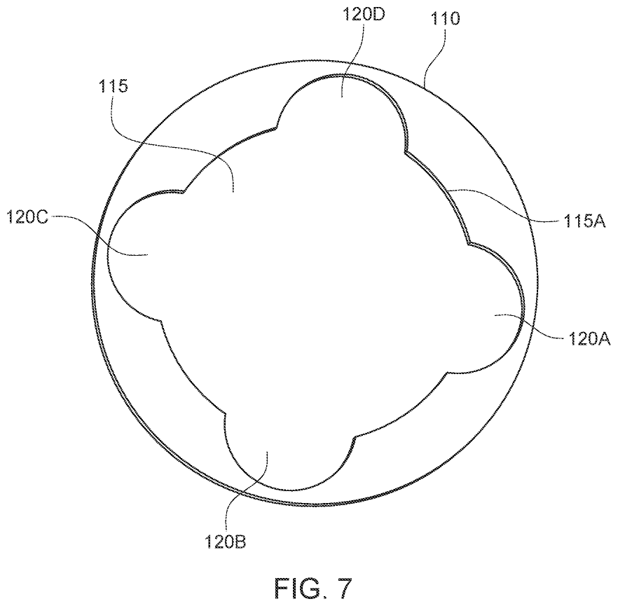

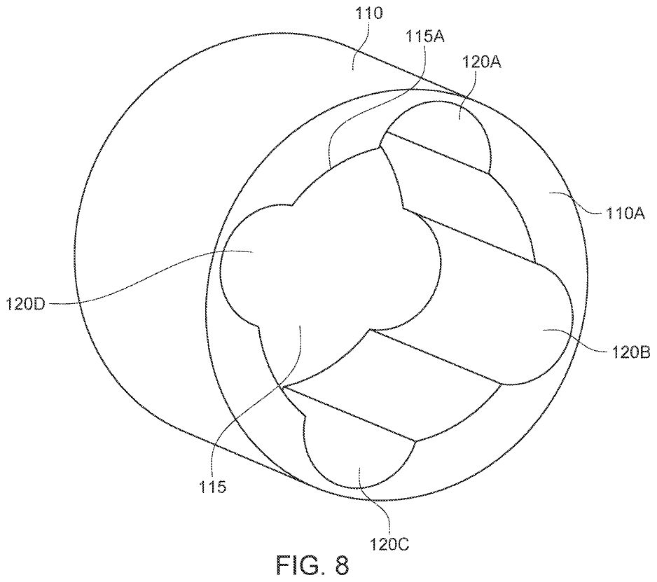

FIG. 7 is an end view of another example multi-cavity aspiration device, according to an example of the present disclosure. FIG. 8 is a partial perspective view of the multi-cavity aspiration device of FIG. 7, according to an example of the present disclosure. As shown in FIGS. 7-8, the portions of the inner circumference 115A of the lumen 115 which separate the cavities 120A, 120B, 120C, 120D may be scalloped or curved. Further, each of the cavities 120A, 120B, 120C, 120D may comprise a semi-cylindrical shaped opening.

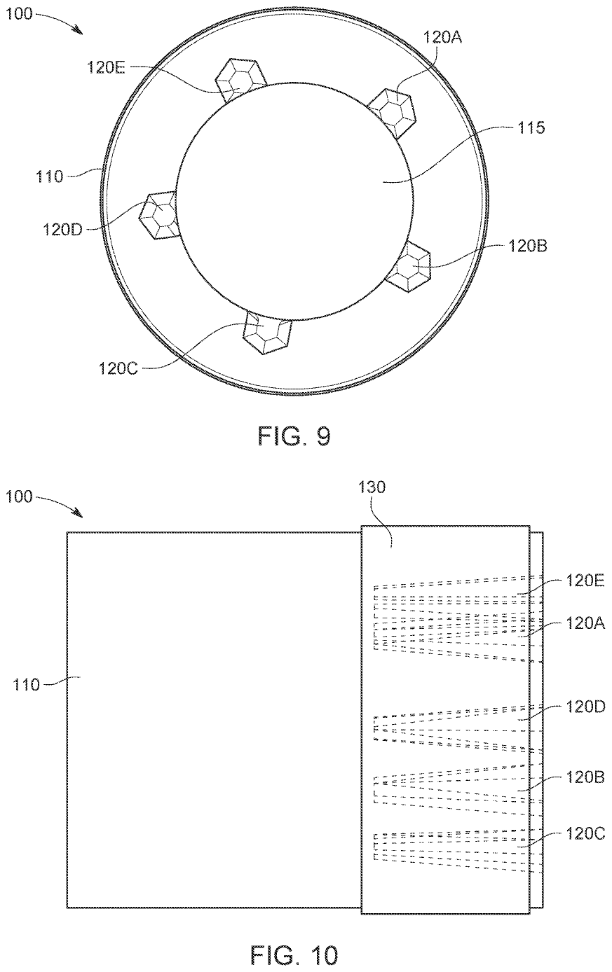

FIG. 9 is an end view of another example multi-cavity aspiration device, according to an example of the present disclosure. FIG. 10 is a partial side view of the multi-cavity aspiration device of FIG. 9, according to an example of the present disclosure. As shown, five cavities 120A, 120B, 120C, 120D, 120E may be utilized. Each of the cavities 120A, 120B, 120C, 120D, 120E may comprise a pentagonal shape having five distinct sides as shown in FIG. 9, with the width or diameter of each of the cavities 120A, 120B, 120C, 120D, 120E reducing in the proximal direction as shown in FIG. 10.

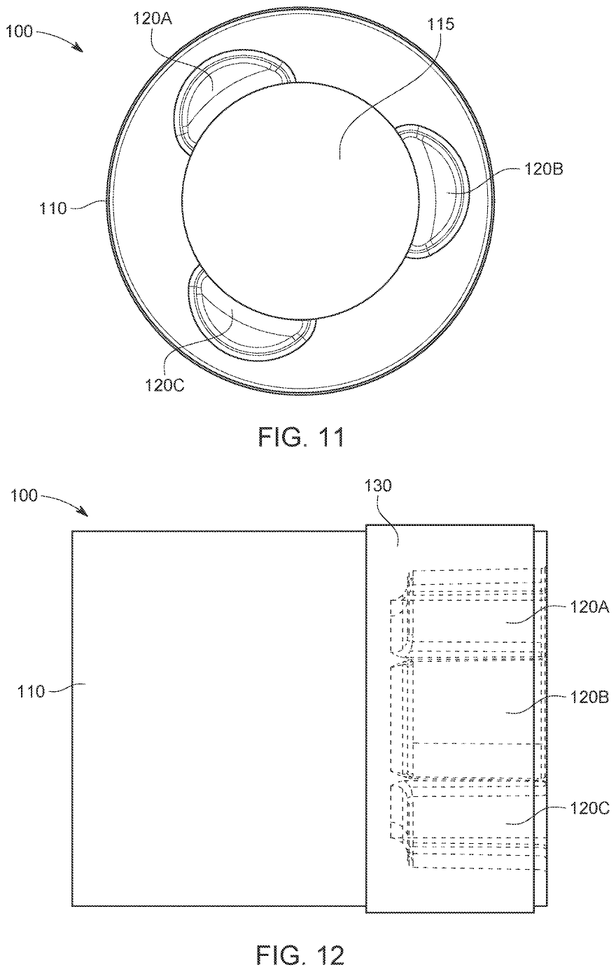

FIG. 11 is an end view of another example multi-cavity aspiration device, according to an example of the present disclosure. FIG. 12 is a partial side view of the multi-cavity aspiration device of FIG. 11, according to an example of the present disclosure. As shown, three cavities 120A, 120B, 120C may be utilized. Each of the cavities 120A, 120B, 120C may comprise a semi-ovular shape as shown in FIG. 11, with the width or diameter of each of the cavities 120A, 120B, 120C being substantially uniform as shown in FIG. 12.

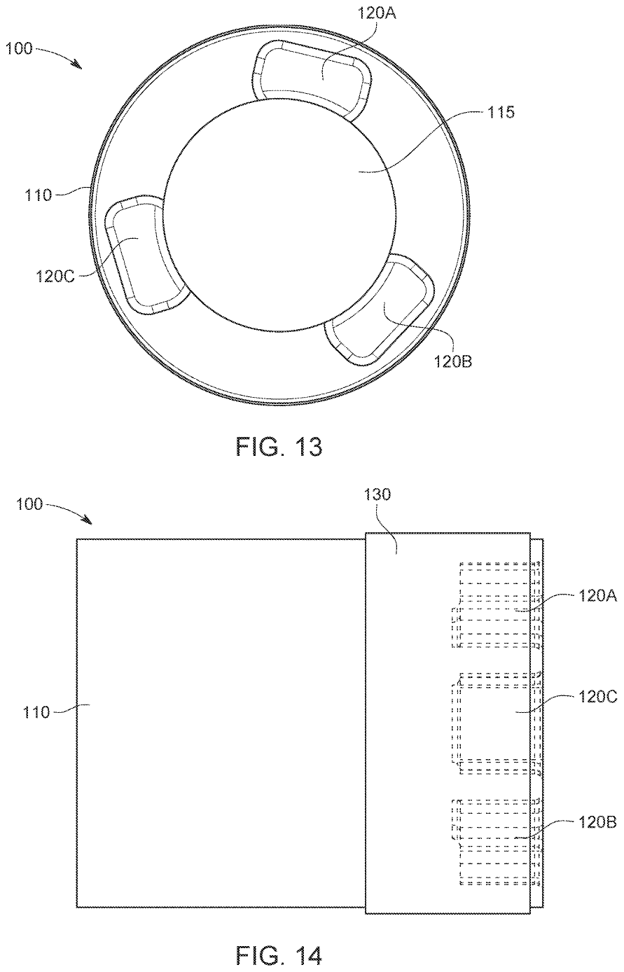

FIG. 13 is an end view of another example multi-cavity aspiration device, according to an example of the present disclosure. FIG. 14 is a partial side view of the multi-cavity aspiration device of FIG. 12, according to an example of the present disclosure. As shown, three cavities 120A, 120B, 120C may be utilized. Each of the cavities 120A, 120B, 120C may comprise an irregular shape which is substantially rectangular with curved corners as shown in FIG. 13, with the width or diameter of each of the cavities 120A, 120B, 120C being substantially uniform as shown in FIG. 14.

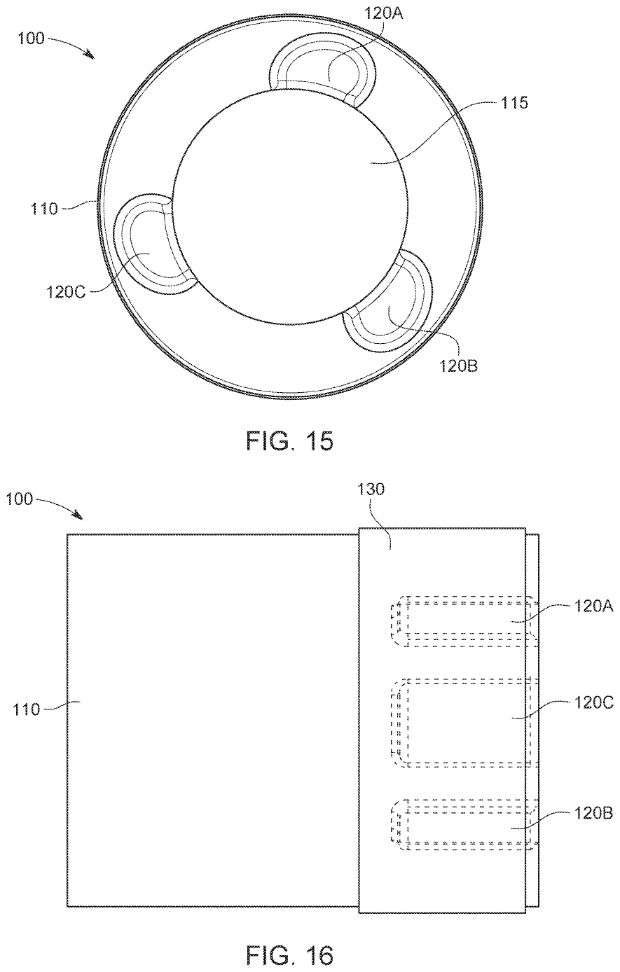

FIG. 15 is an end view of another example multi-cavity aspiration device, according to an example of the present disclosure. FIG. 16 is a partial side view of the multi-cavity aspiration device of FIG. 15, according to an example of the present disclosure. As shown, three cavities 120A, 120B, 120C may be utilized. Each of the cavities 120A, 120B, 120C may comprise an irregular semi-circular shape which is offset in one direction as shown in FIG. 13, with the width or diameter of each of the cavities 120A, 120B, 120C being substantially uniform as shown in FIG. 14.

In some examples, the multi-cavity aspiration device 100 may include reinforcement so as to improve or increase the structural integrity of the tubular device 110 when under suction during aspiration. The types of structural members may vary in different examples. For example, the structural member(s) may comprise ribs, coils, wires, or the like.

As an example, one or more structural members may be positioned along the inner circumference of the tubular member 110 to increase the structural integrity of the aspiration device 100 during aspiration. In some examples, the inner circumference of the tubular member 110 can be reinforced with one or more structural members to prevent the multi-cavity aspiration device 100 from buckling (e.g., local buckling, inward buckling), bending, and/or distorting of the tubular member 110. In some examples, such structural member(s) may be integrated within the wall or jacket of the tubular member 110.

In some examples, such structural member(s) may be attached to or positioned around an outer circumference of the tubular member 110. FIGS. 1A-3, 10, 12, 14, and 16 illustrate use of a radiopaque marker band 130 which provides the dual functions of both reinforcing the structural integrity of the tubular member 110 and aiding in visualization. The radiopaque marker band 130 may be secured (e.g., bonded, fused, or otherwise attached) to the outer circumference of the tubular member 110.

The positioning of the radiopaque marker 130 with respect to the distal end 110A of the tubular member 110 may vary in different examples. In the examples shown in FIGS. 1A, 1B, 10, 12, 14, and 16, the radiopaque marker band 130 may be positioned over or around at least the portion of the tubular member 110 along which the cavities 120A, 120B, 120C, 120D extend. In the examples shown in FIGS. 2-3, the radiopaque marker band 130 may be positioned proximally with respect to the portion of the tubular member 110 along which the cavities 120A, 120B, 120C, 120D extend.

With reference to FIGS. 1A, 1B, 10, 12, 14, and 16, the radiopaque marker band 130 may be slightly offset proximally with respect to the distal end 110A of the tubular member 110. However, in some examples, the radiopaque marker band 130 may be aligned with the distal end 110A of the tubular member 110.

The dimensions (e.g., the length) of the radiopaque marker band 130 may vary in different examples. Thus, the radiopaque marker band 130 may be substantially equal in length to the underlying cavities 120A, 120B, 120C, 120D, may be longer in length than the underlying cavities 120A, 120B, 120C, 120D, or may be shorter in length than the underlying cavities 120A, 120B, 120C, 120D. In some examples, multiple such radiopaque marker bands 130 may be utilized, either directly adjacent to each other or spaced apart.

The radiopaque marker band 130 may be composed of a wide range of materials, including metals, metal alloys, polymers, and the like. In one example, the radiopaque marker band 130 may be composed of a combination of platinum and iridium, such as 90% platinum and 10% iridium.

### Claim Bank

Clause 1. A multi-cavity aspiration device, comprising: a tubular member including a lumen having an inner circumference; and one or more cavities formed in the inner circumference of the tubular member at a distal end of the tubular member.

Clause 2. The multi-cavity aspiration device of clause 1, wherein each of the one or more cavities is tapered in width or diameter.

Clause 3. The multi-cavity aspiration device of clause 1, wherein each of the one or more cavities is tapered in width or diameter such that a proximal end of each of the one or more cavities has a smaller width or diameter than that of a distal end of each of the one or more cavities.

Clause 4. The multi-cavity aspiration device of clause 1, wherein each of the one or more cavities is comprised of a scallop.

Clause 5. The multi-cavity aspiration device of clause 1, wherein each of the one or more cavities is comprised of a slot.

Clause 6. The multi-cavity aspiration device of clause 1, wherein each of the one or more cavities is comprised of a groove.

Clause 7. The multi-cavity aspiration device of clause 1, wherein each of the one or more cavities is comprised of an elongated opening.

Clause 8. The multi-cavity aspiration device of clause 1, wherein the one or more cavities is comprised of a plurality of cavities.

Clause 9. The multi-cavity aspiration device of clause 1, wherein each of the plurality of cavities is equally spaced about the inner circumference of the lumen.

Clause 10. The multi-cavity aspiration device of clause 9, wherein each of the plurality of cavities is comprised of substantially the same dimensions.

Clause 11. The multi-cavity aspiration device of clause 9, wherein the plurality of cavities is comprised of four cavities.

Clause 12. The multi-cavity aspiration device of clause 11, wherein each of the four cavities is positioned at ninety degree intervals along the inner circumference of the lumen.

Clause 13. The multi-cavity aspiration device of clause 1, wherein the one or more cavities extend for only a portion of a length of the tubular member.

Clause 14. The multi-cavity aspiration device of clause 1, further comprising a radiopaque marker band positioned at or near a distal end of the tubular member over the one or more cavities.

Clause 15. The multi-cavity aspiration device of clause 14, wherein a length of the radiopaque marker band is substantially equal to a length of the one or more cavities.

Clause 16. The multi-cavity aspiration device of clause 1, wherein the one or more cavities terminate at a point.

Clause 17. The multi-cavity aspiration device of clause 1, wherein the one or more cavities have a semi-circular cross-section.

Clause 18. A multi-cavity aspiration system, comprising: an aspiration catheter comprising: a tubular member including a lumen having an outer circumference and an inner circumference; one or more cavities formed in the inner circumference of the tubular member at or near a distal end of the tubular member; and a radiopaque marker band attached to the outer circumference of the tubular member over the one or more cavities; and, an aspiration source connected to a proximal end of the aspiration catheter.

Clause 19. The multi-cavity aspiration system of clause 18, wherein the aspiration source is comprised of a syringe.

Clause 20. The multi-cavity aspiration system of clause 18, wherein the aspiration source is comprised of a pump.

Clause 21. A medical device, comprising: a catheter having a proximal portion, a distal portion, and a lumen therebetween, and wherein the catheter has an inner diameter, an outer diameter, and a catheter sidewall therebetween; one or more longitudinal cavities within the catheter side wall at the distal portion of the catheter that are in fluid communication with the lumen and extend to a distal tip of the catheter; and a radiopaque marker band positioned around the distal portion of the catheter over the one or more longitudinal cavities.

Although the disclosure has been described in terms of particular examples and applications, one of ordinary skill in the art, in light of this teaching, can generate additional examples and modifications without departing from the spirit of or exceeding the scope of the claimed disclosure. Accordingly, it is to be understood that the drawings and descriptions herein are proffered by way of example to facilitate comprehension of the disclosure and should not be construed to limit the scope thereof.

1. A multi-cavity aspiration device, comprising:
a tubular member including a lumen having an inner circumference, the lumen having a uniform diameter along its length; and
one or more cavities formed in the inner circumference of the tubular member at a distal end of the tubular member.

2. The multi-cavity aspiration device of claim 1, wherein each of the one or more cavities is tapered in width or diameter.

3. The multi-cavity aspiration device of claim 1, wherein each of the one or more cavities is tapered in width or diameter such that a proximal end of each of the one or more cavities has a smaller width or diameter than that of a distal end of each of the one or more cavities.

4. The multi-cavity aspiration device of claim 1, wherein each of the one or more cavities is comprised of a scallop.

5. The multi-cavity aspiration device of claim 1, wherein each of the one or more cavities is comprised of a slot.

6. The multi-cavity aspiration device of claim 1, wherein each of the one or more cavities is comprised of a groove.

7. The multi-cavity aspiration device of claim 1, wherein each of the one or more cavities is comprised of an elongated opening.

8. The multi-cavity aspiration device of claim 1, wherein the one or more cavities is comprised of a plurality of cavities.

9. The multi-cavity aspiration device of claim 1, wherein each of the one or more cavities is equally spaced about the inner circumference of the lumen.

10. The multi-cavity aspiration device of claim 9, wherein each of the one or more cavities is comprised of substantially the same dimensions.

11. The multi-cavity aspiration device of claim 9, wherein the one or more cavities is comprised of four cavities.

12. (canceled)

13. The multi-cavity aspiration device of claim 1, wherein the one or more cavities extend for only a portion of a length of the tubular member.

14. The multi-cavity aspiration device of claim 1, further comprising a radiopaque marker band positioned at or near a distal end of the tubular member over the one or more cavities.

15. The multi-cavity aspiration device of claim 14, wherein a length of the radiopaque marker band is substantially equal to a length of the one or more cavities.

16. The multi-cavity aspiration device of claim 1, wherein the one or more cavities terminate at a point.

17. The multi-cavity aspiration device of claim 1, wherein the one or more cavities have a semi-circular cross-section.

18. A multi-cavity aspiration system, comprising:
an aspiration catheter comprising:
a tubular member including a lumen having an outer circumference and an inner circumference;
one or more cavities formed in the inner circumference of the tubular member near a distal end of the tubular member; and
a radiopaque marker band attached to the outer circumference of the tubular member over the one or more cavities; and,
an aspiration source connected to a proximal end of the aspiration catheter.

19. The multi-cavity aspiration system of claim 18, wherein the aspiration source is comprised of a syringe.

20. (canceled)

21. A medical device, comprising:
a catheter having a proximal portion, a distal portion, and a lumen therebetween, and wherein the catheter has an inner diameter, an outer diameter, and a catheter sidewall therebetween; and
one or more longitudinal cavities within the catheter sidewall at the distal portion of the catheter that are in fluid communication with the lumen; and
a radiopaque marker band positioned around the distal portion of the catheter over the one or more longitudinal cavities.

22. The medical device of claim 21, wherein the one or more longitudinal cavities extend to a distal tip of the catheter.
